SEQUENCE LISTING (2 sequences)

SEQ ID NO: 1 - protein, 111 aa, Artificial Sequence
  Description of artificial sequence Synthetic Peptide Apo B100
KAASGTTGTYQEWKDKAQNLYQELLTQEGQASFQGLKDNVFDGLVRVTQKFHMKVKHLIDSLIDFLNFPRFQFPGKPGIYTREELSTMFIREVGTVLSQVYSKVHNGSEIL

SEQ ID NO: 2 - protein, 108 aa, Artificial sequence
  Description of artificial sequence synthetic Peptide Apo B100
AFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYH

(19) United States
(12) Patent Application Publication
(10) Pub. No.: US 2005/0152900 A1
(43) Pub. Date: Jul. 14, 2005

(54) Compositions and methods for apo-b48 and apo-b100 assay

(75) Inventors: Jamila Najib, Santes (FR); Zouher Majd, Ennetieres en Weppes (FR)

Correspondence Address: NIXON & VANDERHYE, PC, 1100 N GLEBE ROAD, 8TH FLOOR, ARLINGTON, VA, 22201-4714, US

(73) Assignee: GENFIT, Loos (FR)

(21) Appl. No.: 10/513,916

(22) PCT Filed: Jun. 19, 2003
(86) PCT No.: PCT/FR03/01888

(30) Foreign Application Priority Data
Jun. 19, 2002 (FR) 02/07542

(51) Int. Cl. A61K 39/395; C07K 16/18

(52) U.S. Cl. 424/145.1; 530/350; 530/388.25; 530/359

(57) ABSTRACT

The invention concerns compositions and methods for assay or detection of apolipoprotein-B48 in samples. In particular, it concerns a method for differential measurement of apolipoprotein-B48 (“Apo-B48”) and apolipoprotein-B100 (“Apo-B100”) in biological samples. The invention also concerns synthetic products of Apo-B100, the corresponding antibodies, kits containing same, and their uses for detecting, differentially quantifying and/or recording an amount of Apo-B48 and/or Apo-B100 in a sample, or for quantifying and/or recording atherogenic lipoparticles in a sample. The products, materials and kits hereinabove can also be used for differentially modulating the levels of Apo-B48 and/or Apo-B100 or their activity, in vitro or in vivo, and for regulating lipid metabolism in a subject.

### INTRODUCTION AND PRIOR ART

The invention concerns compositions and methods for assay or detection of apolipoprotein-B48 or apolipoprotein-B100 in samples. In particular, it concerns a method for detection and differential quantification of apolipoprotein-B48 (“Apo-B48”) and apolipoprotein-B100 (“Apo-B100”). The invention also concerns synthetic products of Apo-B100, the corresponding antibodies, kits comprising same, and their uses for detecting, quantifying and/or following over time the amounts of Apo-B48 and/or Apo-B100 in a biological sample. The products, materials and kits of the invention can also be used for differentially modulating the levels of Apo-B48 and/or Apo-B100 or their activity, in vitro or in vivo, and for regulating lipid metabolism in a subject.

High triglyceride levels in plasma and persistence of chylomicrons in the diet are risk factors for cardiovascular disease which is currently the leading cause of death worldwide (Hokanson and Austin 1996). A high level of plasma lipids is correlated with the level of Apo-B lipoproteins considered to be atherogenic.

Apolipoproteins B are heterogeneous. Very low density lipoproteins (VLDL: Very Low Density Lipoprotein) and low density lipoproteins (LDL: Low Density Lipoprotein) contain an isoform with an apparent molecular weight of approximately 549,000 Da, which is designated by the abbreviation B100 in a centile nomenclature system, while the major isoform present in chylomicrons (the majority of postprandial lipoproteins) has an apparent molecular weight of 246,000 Da and is identified by the abbreviation B48 in the same nomenclature system. Apo-B48 is a truncated form of Apo-B100 and is produced by post-transcriptional modification of Apo-B100 messenger RNA. Said modification converts codon 2153 (CM, coding for a glutamine) to a stop codon (Davidson, Anant et al. 1995).

Apo-B48 and Apo-B100 are key proteins in lipoprotein metabolism in mammals. Apo-B100 is required for VLDL assembly (Havel 1995) in human liver (Kane 1983) and also plays a role in cellular uptake and catabolism of LDL. It has been shown to participate in the LDL receptor pathway (Goldstein and Brown 1977). Apo-B48 is required for assembly of chylomicrons in the intestine (Young 1990) (Kane 1983) and is characteristic of “remnant” (i.e., residual) particles of chylomicrons, considered to be additional risk factors for coronary disease (Simons, Dwyer et al. 1987).

At the present time, very little information is available about the atherogenic nature of chylomicron particles containing Apo-B48 derived from intestine and Apo-B100-containing particles derived from liver. It is therefore of particular relevance to develop a method by which to detect and/or differentially quantify Apo-B48 and Apo-B100 so as, in particular, to be able to study their respective roles and demonstrate deregulations or dysfunctions in subjects.

For various reasons, it is fairly difficult to differentially measure Apo-B48 and Apo-B100 concentrations in plasma. In the first place, the amino acid sequence of Apo-B48 is identical to the Apo-B100 N-terminal region (accounting for 48% of Apo-B100). Secondly, Apo-B48 is present at very low levels in plasma, in contrast to Apo-B100. Thirdly, the expression of Apo-B epitopes varies according to the lipid content of the particles.

Procedures based on immunological methods and allowing quantification of Apo-B48 have been described in the prior art. In particular, the method of Uchida et al. (Uchida, Kurano et al. 1998) makes use of an ELISA test (Enzyme-Linked Immuno-Sorbent Assay) to quantify Apo-B48. However, the precision and output of this method are quite low hence the need to purify and label the antibodies. Moreover, the monoclonal antibody used is directed against the C-terminal region of human Apo-B48. Said antibody is obtained thanks to the differential conformation of Apo-B48 in Apo-B100-containing paticles, which may lead to antibodies with different specificity and affinity according to the lipid composition of the lipoparticles.

Other documents describe a quantification of Apo-B48 by means of non-immunochemical methods such as capillary electrophoresis (Proctor and Mamo 1997), liquid chromatography on an HPLC system (Hidaka, Kojima et al. 1990) (Wagner, Nustede et al. 1987) or SDS PAGE (Karpe and Hamsten 1994) (Smith, Proctor et al. 1997). Such methods have a number of drawbacks: they have a low output and require lipoprotein preparation and delipidation steps to improve the precision of the results. Such methods are also much less sensitive than immunochemical methods.

### SUMMARY OF THE INVENTION

The invention provides a novel strategy for producing synthetic peptides specific of Apo-B100 and novel methods for detecting and differentially quantifying Apo-B100 and Apo-B48. Differential quantification is typically carried out by assaying total Apo-B (Apo-B100 plus Apo-B48) in a sample, preferably with the help of an anti-Apo-B polyclonal antibody. Apo-B100 is then depleted from the sample with a specific anti-Apo-B100 antibody, so as to directly and specifically quantify Apo-B48.

More particularly, the invention describes novel methods for producing antibodies specific of Apo-B100 through the use of synthetic peptides specific of Apo-B100. The invention also describes said antibodies, kits containing same and uses thereof for detecting, quantifying, purifying and/or monitoring changes in the amounts of Apo-B100 and Apo-B48 in serum or plasma. Said antibodies also offer a novel approach for modulating Apo-B activity in vitro or in vivo, and for regulating lipid metabolism in a subject.

A first particular object of the invention concerns a substantially pure synthetic peptide specific of Apo-B100, comprising sequence SEQ ID NO: 1 or sequence SEQ ID NO: 2, or an immunogenic fragment or a derivative of said peptide.

Another object of the invention concerns a method for producing anti-Apo-B100 antibody comprising an immunization step with the help of a synthetic peptide specific of Apo-B100 such as defined hereinabove. The invention also comprises antibodies prepared according to said method, as well as, more generally, antibodies capable of binding a peptide such as defined hereinabove as well as fragments or derivatives of such antibodies.

Another aspect of the invention concerns a method for detecting or assaying Apo-B100 or Apo-B48 in biological samples (particularly in prepared lipoparticles or in plasma or serum samples), with the help of an antibody (including a fragment or derivative thereof) such as defined hereinabove.

Another object of the invention concerns a method for detecting the presence, a predisposition or a risk of developing a disorder of lipid metabolism in a subject, comprising detecting Apo-B100 and Apo-B48 in vitro, in a sample from said subject, with the help of an antibody (including a fragment or derivative thereof) such as defined hereinabove. The method is particularly suited to detecting the presence, a predisposition or a risk of developing atherosclerosis or a cardiovascular disease such as a coronary disease for instance.

In this respect, a specific object of the invention is a method for detecting and/or monitoring the formation, development or progression of atherosclerosis in a subject, comprising detecting the amount or level of Apo-B100 and/or Apo-B48 in vitro, in a sample from said subject, with the help of an antibody (including a fragment or derivative thereof) such as described hereinabove.

Another object of the invention concerns a method for detecting or monitoring in a subject the cellular uptake of lipoproteins rich in triglycerides and LDL, comprising detection in vitro of particles containing Apo-B with the help of an antibody (including a fragment or derivative thereof) such as defined hereinabove.

Generally, the subject is a mammal, preferably a human being, even more preferably a subject presenting a risk of developing cardiovascular diseases in relation to a disorder of lipid metabolism such as a coronary disease, or else a subject presenting with such disease.

Another object of the invention relates to a pharmaceutical composition comprising an antibody (or a fragment or derivative thereof) such as defined hereinabove and a pharmaceutically acceptable vehicle or excipient.

### DETAILED DESCRIPTION OF THE INVENTION

As indicated earlier, one aspect of the invention concerns a substantially pure synthetic peptide specific of Apo-B100, comprising sequence SEQ ID NO: 1 or sequence SEQ ID NO: 2, or an immunogenic fragment or a derivative of said peptide. “Synthetic peptide specific of Apo-B100” is understood to mean all artificially synthesized peptides mimicking part of the native Apo-B100 protein and containing regions or epitopes specific to said protein and essentially absent from other proteins. In particular, it is a peptide comprising the sequence of a region of Apo-B100 absent in other Apo-B forms, particularly in Apo-B48. The term “substantially pure” indicates that the peptide is essentially free of amino acid sequences present in Apo-B48 and/or of contaminating proteins normally present or associated with Apo-B in lipoparticles, such as Apo-A1 in particular. The term “synthetic” indicates that the peptide is not a molecule obtained naturally but that it has been prepared by artificial means (eg., chemical synthesis, assembly, and the like) particularly as described in the examples. In this context, the synthetic peptide specific of Apo-B100 of the invention preferably is essentially non-glycosylated.

The invention now shows that synthetic peptides specific of Apo-B100 can be produced and used to generate specific anti-Apo-B100 antibodies. The invention further shows that said antibodies are capable of binding specifically to Apo-B100 obtained either by natural means or in the form of a soluble antigen, or included in lipoparticles. The invention also shows that said antibodies can bind to different lipoparticles containing Apo-B100 (IDL (Intermediary Density Lipoprotein), VLDL and LDL), and display immunoprecipitation properties. Said synthetic peptides specific of Apo-B100 and their corresponding antibodies thus represent novel products which are particularly advantageous for detecting Apo-B100 and for differentially quantifying Apo-B100 and Apo-B48.

More particularly, a preferred synthetic peptide specific of Apo-B100 according to the invention comprises the sequence SEQ ID NO: 1, such as described below, or an immunogenic fragment or a derivative of said peptide.

| | | |
|---|---|---|
| SEQ ID NO: 1: | | |
| KAASGTTGTY | QEWKDKAQNL | YQELLTQEGQ |
| ASFQGLKDNV | FDGLVRVTQK | FHMKVKHLID |
| SLIDFLNFPR | FQFPGKPGIY | TREELSTMFI |
| REVGTVLSQV | YSKVHNGSEI | L |

Said peptide corresponds to residues 4105 to 4215 of the mature human Apo-B sequence.

In a variant of the invention, a preferred synthetic peptide specific of Apo-B100 according to the invention comprises the sequence SEQ ID NO: 2, such as described below, or an immunogenic fragment or a derivative of said peptide.

| | | |
|---|---|---|
| SEQ ID NO: 2: | | |
| AFTDLHLRYQ | KDKKGISTSA | ASPAVGTVGM |
| DMDEDDDFSK | WNFYYSPQSS | PDKKLTIFKT |
| ELRVRESDEE | TQIKVNWEEE | AASGLLTSLK |
| DNVPKATGVL | YDYVNKYH | |

Said peptide corresponds to residues 3955 to 4062 of the mature human Apo-B sequence.

The peptides accodring to the invention advantageously contain less than 200 amino acids, more preferably less than 180 amino acids.

As illustrated in the examples, the peptides of the invention (comprising sequence SEQ ID NO: 1 or sequence SEQ ID NO: 2) can be prepared in a particularly advantageous manner by solid phase synthesis, more particularly by using a Boc/Bzl strategy (Merrifield 1963).

The term “derivative” includes peptides comprising one or more mutations, substitutions, deletions and/or additions of one or more amino acid residues and having substantially the same antigenic specificity. Typical examples of derivatives include sequence variations due to Apo-B polymorphisms, splicing, etc. Especially preferred derivatives comprise a modified sequence SEQ ID NO: 1 or sequence SEQ ID NO: 2 comprising at most 5 amino acid residues different from those present in SEQ ID NO: 1 or in SEQ ID NO: 2. The additional residues may correspond to residues of transport or binding sequences, protective groups, etc. In addition, the peptide may be modified, for example, by a chemical, physical and/or enzymatic route, so as to increase its stability, boost its immunogenicity or else incorporate a tag or label, etc. Examples of such modifications include glycosylation, addition of a tag (eg., myc), label (eg. radioactive or enzymatic labelling, etc.), and the like.

A particular object of the invention concerns a synthetic peptide specific of Apo-B wherein it comprises epitopes specific of Apo-B100 and wherein it is free of epitopes specific of Apo-B48, and wherein it comprises sequence SEQ ID NO: 1 or sequence SEQ ID NO: 2 or a fragment or derivative of said sequences.

The term “fragment” denotes any peptide comprising at least 5, preferably between 5 and 20, consecutive residues of segments 4105-4139 and 4198-4215 of sequence SEQ ID NO: 1 or any peptide comprising at least 5, preferably between 5 and 40, consecutive residues of segment 4022-4062 of sequence SEQ ID NO: 2. The term “fragment” includes any portion such as described hereinabove of sequence SEQ ID NO: 1 or sequence SEQ ID NO: 2 comprising an epitope, preferably comprising at least 10 consecutive residues of the abovedescribed segments.

The peptides may be soluble, purified or complexed with a carrier molecule, such as KLH (Keyhole Limpet Hemocyanin) or serum albumin for example, or else with any other inert molecule (eg., synthetic) such as a bead, and the like. According to a particular embodiment of the invention, the peptides are coupled with a carrier molecule. This is particularly the case when they are used to produce antibodies. The coupling may be carried out according to conventional methods known to those skilled in the art (Vaitukaitis, Robbins et al. 1971) (Bassiri 1979).

The peptides may also be conjugated or coupled with a heterologous polypeptide molecule, such as a biologically active molecule for example. The hereologous nature designates any peptide that does not originate from a human Apo-B molecule.

A particular object of the invention concerns a composition characterized in that it comprises a synthetic peptide comprising sequence SEQ ID NO: 1 or sequence SEQ ID NO: 2, and that it is free of other apolipoprotein.

The peptides may be used in methods for screening compounds that modulate Apo-B activity, titration methods, as controls, standards or to calibrate analytical methods. They may also be used for modulating Apo-B100 activity, advantageously in a specific manner. They are also particularly useful for producing anti-Apo-B100 antibodies.

In this respect, another object of the invention concerns any antibody capable of binding to a peptide such as defined hereainbove, preferably in a specific manner. Preferably, the inventive antibodies have the capacity to precipitate Apo-B100 (immunoprecipitant). The antibody may be polyclonal or monoclonal. Advantageously, when the antibodies are monoclonal, they are present in the form of a mixture of monoclonal antibodies. In addition, the term antibody further includes any antibody fragments and derivatives, in particular fragments and derivatives of said monoclonal or polyclonal antibodies having substantially the same antigenic specificity. The latter comprise antibody fragments (eg., Fab, F(ab′)2, CDRs, etc.), humanized antibodies, polyfunctional antibodies, monocatenary antibodies (ScFv), and the like. The inventive antibodies may be produced by conventional methods, comprising immunizing an animal and recovering the serum (polyclonal) or spleen cells (so as to produce hybridomas by fusion with suitable cell lines).

Methods for producing polyclonal antibodies from various animal species including rodents (mice, rats, etc.), primates, horses, pigs, sheep, rabbits, fowl and the like are described for example in Vaitukaitis et al. (Vaitukaitis, Robbins et al. 1971). The antigen is combined with an adjuvant (eg., Freund's adjuvant) and administered to an animal, typically by subcutaneous injection. Repeated injections may be given. Blood samples (immune serum) are collected and the immunoglobulins are isolated.

Methods for producing monoclonal antibodies from different animal species may be found for example in Harlow et al. (Harlow 1988) or Kohler et al. (Kohler and Milstein 1975). Said methods comprise immunizing an animal with an antigen, then recovering the spleen cells which are fused with immortalized cells, such as myeloma cells. The resulting hybridomas produce monoclonal antibody and may be selected by limit dilution so as to isolate individual clones. The antibodies may also be produced by selection of immunoglobulin combinatorial libraries, such as those disclosed for example in Ward et al. (Ward, Gussow et al. 1989).

Preferred antibodies of the invention are prepared by immunizing a non-human animal with a substantially pure synthetic peptide specific of Apo-B100 such as described hereinabove, preferably comprising sequence SEQ ID NO: 1 or sequence SEQ ID NO: 2, or an immunogenic fragment or a derivative of said peptide, eg., a subfragment containing at least one epitope, or a mixture of immunogenic fragments or derivatives of SEQ ID NO: 1 and SEQ ID NO: 2 (oligopeptides) and recovering the antibodies or antibody-producing cells.

Fab or F(ab′)2 fragments may be produced by digestion with a protease according to conventional techniques. Humanized antibodies may be prepared by one of the methods described, for example, in Riechmann et al. (Riechmann 1988) (Jones 1986).

The invention also concerns a method for producing specific anti-Apo-B100 antibodies, comprising administering (eg., injecting) a peptide with sequence SEQ ID NO: 1 or sequence SEQ ID NO: 2, or an immunogenic fragment or a derivative of said peptide, such as described hereinabove, to a non-human animal and recovering the antibodies or antibody-producing cells.

The method advantageously enables the production of specific and immunoprecipitant antibodies. Specificity can be verified by demonstrating an absence of cross reaction with other circulating proteins in blood comprising Apo-B48. More generally, specificity indicates that the antibodies are capable of binding to Apo-B100 with a higher affinity than to any other antigen. As illustrated in the examples, the polyclonal antibodies of the invention are immunoprecipitant, and thus can be used to detect Apo-B100 or to differentially assay Apo-B100 and Apo-B48 with high efficiency.

The antibodies may be coupled with heterologous fragments such as toxins, labels, medicaments or any other therapeutic agent, covalently or not, either directly, or by means of coupling agents. Labels may be selected in the group consisting of radiolabels, enzymes, fluorescent labels, magnetic particles, etc. Preferred toxins are for example the diphtheria toxin, botulism toxin, etc. Medicaments or therapeutic agents are selected in the group consisting in particular of lymphokines, antibiotics, antisense sequences, growth factors, etc. Methods by which to carry out the coupling of antibodies and such heterologous fragments are described in U.S. Pat. No. 4,277,149 and U.S. Pat. No. 3,996,345 for example.

The antibodies according to the invention have many uses including in therapeutics, prophylaxis, diagnostics, purification, detection and the like.

They can be used in vitro as screening agents or to purify antigens from various samples, including various biological samples (eg., blood samples). They can also be used to detect or quantify the presence or amount of Apo-B100 or indirectly of Apo-B48 in Apo-B-containing lipoparticles present in a sample collected from a subject, typically a blood sample from a mammal or, preferably, a human being.

In this respect, another object of the invention concerns a method for detecting or quantifying Apo-B100, in a biological sample, comprising contacting the sample with an antibody such as defined hereinabove (including fragments or derivatives thereof) and detecting the presence of antigen-antibody immune complexes.

Quantification is typically carried out in a differential manner, that is, by assaying total Apo-B (Apo-B100 plus Apo-B48) in a sample, which is then depleted of Apo-B100 by means of a specific anti-apo-B100 antibody, allowing direct and specific quantification of Apo-B48. The Apo-B100 concentration is obtained by the difference between the total Apo-B measurement and that of Apo-B48. Said method therefore makes it possible to determine Apo-B100 and Apo-B48 levels in a sample, by determining the (relative) amounts of immune complexes in the sample (before and after recovering Apo-B100) and comparing this to standard conditions or to a calibration curve for example.

A preferred method of detection or differential quantification therefore advantageously comprises the following steps:
a. determining the total amount of Apo-B in a sample with a total anti-Apo-B antibody,
b. eliminating Apo-B100 or lipoparticles containing Apo-B100 from said sample, with the help of specific anti-Apo-B100 antibodies such as described hereinabove, and
c. determining the amount of Apo-B48 in the sample obtained after step (b) or in lipoparticles that do not contain Apo-B100 in said sample, with a total anti-Apo-B antibody.

By deduction, the amounts obtained in (a) and (c) allow to determine the amounts of Apo-B100 (or of lipoparticles containing Apo-B100) in the sample.

The antibodies used may be polyclonal, monoclonal or fragments or derivatives of same, alone, modified and/or in a mixture. In particular, they may be used in soluble form or immobilized on supports, particularly beads, plates, columns and the like.

The total Apo-B antibody is advantageously composed of a mixture of monoclonal antibodies or a polyclonal antibody, so as to ensure complete detection or quantification of the antigen, in its different forms. Furthermore, the antibody used in steps (a) and (c) may be identical or different. Generally, it is the same antibody.

Typically, a polyclonal antibody is used as total anti-Apo-B antibody. The total anti-Apo-B polyclonal antibody may be obtained by any method known to those skilled in the art, particularly by immunization with whole Apo-B. Such antibodies are described in particular in the publications of Li (Li, Wu et al. 1997) and Levinson (Levinson and Wagner 1993).

Advantageously, during step (b), in order to selectively eliminate the Apo-B100 present in the sample, one uses one or more specific anti-Apo-B100 antibodies according to the invention, the binding of which to Apo-B100 induces precipitation of the complex. As non-limiting examples, it is therefore possible to use either polyclonal antibodies, a mixture of monoclonal antibodies, or antibodies (polyclonal or monoclonal) modified so as to induce precipitation of the Apo-B100-antibody immune complexes.

In a first variant, the specific anti-Apo-B100 antibodies used in step (b) are polyclonal antibodies, having the immunoprecipitant property.

In another variant, it is also possible to use a mixture of specific anti-Apo-B monoclonal antibodies or modified monoclonal antibodies (for example bound to latex or other particles) so as to induce precipitation of the Apo-B-antibody immune complexes.

Another way to eliminate lipoproteins containing Apo-B100 is to bind specific anti-Apo-B100 antibodies to A or G proteins or to other chemical molecules (chemical polymers or resins such as polyurethane, activated sepharose, etc.). Said complexes may be fixed to magnetic beads, allowing elimination of Apo-B100 by application of a magnetic field (eg., magnetic separation or precipitation).

The analytical method according to the invention (and more specifically the detection or quantification of antigen-antibody immune complexes) may be carried out by means of conventional technic such as ELISA methods (direct or competitive immunoassay), RIA (Radio ImmunoAssay), EIA (Electro immunoassay also called Electro immunodiffusion), turbidimetry, or any other immunological method. However, a particularly preferred object of the invention concerns a nephelometric method of analysis. In fact, as indicated earlier, the antibodies are specific and can immunoprecipitate Apo-B100 in a sample thereby allowing differential assay of Apo-B48 with a total anti-Apo-B polyclonal antibody (or a mixture of monoclonal antibodies or a monoclonal antibody modified so as to induce precipitation of Apo-B-antibody immune complexes).

In the nephelometric analytical method, the intensity of the light emitted by the particles in suspension is measured on an analyzer. The particles are formed during the immunoprecipitation reaction which occurs, in buffer medium stimulating polymer formation, when a specific antibody contacts a specific antigen. The complex comprising an antigen and an antibody specific of the antigen forms at a level which gradually increases over time, then rises rapidly and finally reaches a peak value which is proportional to the antigen concentration. The analytical method is based on measuring the maximum rate of change in the light signal intensity which is correlated with (and can be converted to) antigen concentration. Typically, the nephelometric method of analysis is carried out with the help of Beckman immunochemical systems (IMMAGE, Beckman Coulter, Foster City, Calif., USA) which present the results on an alphametric table. The nephelometric method of the invention is particularly advantageous in so far as it is rapid and reproducible and has a high output. In fact, said analytical method requires only a few seconds per sample, while the methods of the prior art, by way of comparison, require a full day. Furthermore, the method of the invention can be easily automated. In addition, the coefficient of variation for Apo-B detection (irrespective of isoform) is only 4% in the inventive method as compared with 10% in the prior art.

A particular object of the invention thus concerns a method for differentially quantifying Apo-B48 and Apo-B100 in lipoparticles present in a biological sample, comprising contacting the sample (or a dilution of same) with an antibody such as described hereinabove (including fragments or derivatives thereof). Briefly, in a first step, a biological sample is assayed with total anti-Apo-B antibody in an immunonephelometric system thereby allowing quantification of total circulating Apo-B. In a second step the biological sample is depleted of the Apo-B100 therein by contacting said sample with a specific anti-Apo-B100 antibody (preferably produced through the use of peptides having SEQ ID NO: 1 or SEQ ID NO: 2). Lastly the amount of Apo-B48 in the Apo-B100-depleted sample is assayed with the total anti-Apo-B antibody in the same manner as the crude sample. The amount of Apo-B100 can then be obtained simply as the difference between the amount of total Apo-B and the amount of Apo-B48. Typically the different dilutions of the biological sample and/or the total anti-Apo-B antibodies and/or the anti-Apo-B100 antibody are subjected to a treatment prior to contacting with the sample, with a view to eliminating non-immunoglobulin proteins and/or concentrating the antibody. The treatment typically comprises contacting the antibodies with polyethylene glycol (PEG), such as described for example in Ritchie et al. (Ritchie 1972). Typically, 0.5 to 1 μg of specific antibodies are used in this analytical method, although those skilled in the art may use different amounts without significantly deviating from the invention.

In a nephelometric analytical method, polyclonal antibodies are generally used.

The analytical method may be carried out on different biological samples, including in particular blood, plasma, serum, interstitial fluid, cerebrospinal fluid, cell culture supernatant, and the like. The sample may be collected from a subject (eg., a human subject) and used directly to practice the analytical method. Alternatively, the sample may be diluted and/or stored (for example frozen) for testing at a later date. The invention also provides for measurement of Apo-B48 and Apo-B100 concentrations in lipoparticles by means of total anti-Apo-B and specific anti-Apo-B100 antibodies, with high output, efficiency and safety.

Detection may be carried out in different experimental, clinical and/or diagnostic conditions. In particular, the method may be used to detect the predisposition of certain individuals to develop disorders of lipid metabolism.

A particular object of the invention thus concerns a method for detecting the presence, a predisposition or a risk of developing a disorder of lipid metabolism in a subject, comprising detecting in vitro or quantifying in vitro (or ex vivo), in a sample taken from the subject, lipoparticles comprising Apo-B48 and Apo-B100, with the help of an antibody such as defined hereinabove (including fragments or derivatives thereof). Elevated Apo-B48 and/or Apo-B100 levels (compared with a mean value in normal subjects) are characteristic of this increased risk of developing disorders of lipid metabolism. Said method is particularly suited to detecting the presence, a predisposition or a risk of developing atherosclerosis or a cardiovascular disease such as a coronary disease.

Another object of the invention concerns a method for detecting or monitoring the cellular uptake of LDL and triglyceride-rich lipoproteins in a subject, comprising detecting in vitro the amounts of Apo-B48 and/or Apo-B100 present in lipoparticles, with the help of an antibody such as defined hereinabove (including fragments or derivatives thereof).

Another object of the invention concerns a method for monitoring a treatment aimed at correcting disorders of lipid metabolism in a subject, comprising measuring in vitro, preferentially in a differential manner, the amounts of Apo-B48 and/or Apo-BOO in a sample from said subject, with the help of an antibody such as defined hereinabove (including fragments or derivatives thereof), after administering said treatment to said subject. The efficacy of the treatment is correlated with the level of Apo-B48 and/or Apo-B100 in the subject. These results can be correlated with the capacity of the treatment to regulate the amounts or the activity of Apo-B48 and/or Apo-B100 in a subject. Said method is particularly adapted to monitoring a treatment of atherosclerosis or of a cardiovascular disease such as a coronary disease for example.

Another object of the invention concerns a method for evaluating the physiological state of a subject, eg., the level of lipid metabolism in a subject, comprising detecting Apo-B48 and/or Apo-B100 levels in vitro in a sample from said subject, by means of an antibody such as defined hereinabove (including fragments or derivatives thereof).

The antibodies according to the invention may also be used for screening (selecting) compounds or diets that are capable of modulating the concentration of Apo-B48 and/or Apo-B100 in serum or plasma. Typically, the method comprises administering to an animal or a patient a compound or a diet and recovering a biological sample from the animal or patient, then detecting the presence and/or assaying the amounts of Apo-B48 and Apo-B100 in said sample with the help of an antibody such as defined hereinabove (including fragments or derivatives thereof).

As noted earlier, said methods may be carried out on different samples (typically plasma or serum) and by ELISA, RIA, EIA, turbidimetry, etc. or preferably by means of a nephelometric method of analysis.

Another object of the invention concerns the use of an antibody according to the invention for preparing a composition designed to selectively regulate Apo-B100 activity in vivo in a subject. For example in order to inhibit the uptake of Apo-B100-containing lipoproteins (via the LDL receptor) by liver or peripheral cells, so as to modify the supply of lipids thereto, or to inhibit intracellular assembly and secretion of lipoproteins (since Apo-B100 is needed for such assembly), thereby decreasing the production of so-called atherogenic lipoproteins.

In particular, Apo-B100 activity is understood to mean its role in the assembly and secretion of lipoproteins in plasma (VLDL) and its role in lipoprotein uptake by peripheral cells.

The invention further comprises a pharmaceutical composition comprising an antibody such as defined hereinabove and optionally a pharmaceutically acceptable vehicle or excipient. The excipient may be any solution, suspension, gel, powder, etc. compatible with a pharmaceutical use. Isotonic solutions, buffers, saline solutions, etc. possibly combined with stabilizers, such as proteins or other high molecular weight molecules, may be cited as examples.

The invention also relates to a kit comprising a peptide or an antibody such as defined hereinabove. The kit may be used for detecting or quantifying Apo-B48 and/or Apo-B100 in any sample.

Other aspects and advantages of the invention will be described in the following examples, which are given for purposes of illustration and not by way of limitation of the invention.

### LEGENDS OF FIGURES

FIG. 1: Specificity of anti-Apo-B100 antibody. A: SDS PAGE; B ImmunoBlot. Lanes 1 and 2: Chylomicrons from HTG subjects. Lane 3: VLDL from normolipemic subjects.

FIG. 2: Nephelometric method of Apo-B analysis: calibration curve.

### EXAMPLES

1. Apo-B100 Synthesis

Synthetic peptides specific of Apo-B100 were synthesized by a solid phase method (Merrifield 1963) on a model ABI 431 automatic synthesizer (Applied Biosystems Inc. Foster City, Calif., USA) using a Boc/Bzl strategy, on 0.5 mmol of PAM-Ala resin. Each amino acid was coupled twice by using dicyclohexylcarbodiimide/hydroxybenzotriazole without capping.

Crude products were then purified and analyzed by reverse phase HPLC on a Vydac 18 column (Interchim, Montlucon, France) using a linear gradient from 0 to 100% buffer B (buffer A: 0.05% TFA in H₂O and buffer B: 0.05% TFA, with 60% CH₃CN in H₂O). Molecular masses were then determined on an API mass spectrometer (Perkin-Elmer, Foster City, Calif., USA) equipped with a single quadripole and electrospray ion source (nebulizer-assisted electrospray) (Sciex, Toronto, Canada).

Amino acid analysis was performed on a Beckman 6300 amino acid analyzer (Beckman Instruments, Fullerton, Calif., USA), after hydrolysis in 6 N HCl containing 0.25% phenol at 110° C. for 24 hours.

2. Immunizations

An antiserum directed against total Apo-B was prepared as described by Fievet et al. (Fievet 1984). Briefly, LDL with a density between 1.030 and 1.053 (therefore containing only Apo-B100) were prepared by ultracentrifugation, then injected into goats so as to produce total anti-Apo-B immune serum able to recognize all Apo-B isoforms.

Specific Apo-B100 antiserum was also prepared in goats essentially as described in the protocol of Vaitukaitis et al. (Vaitukaitis, Robbins et al. 1971). The peptide (SEQ ID NO: 1 or SEQ ID NO: 2) which served as antigen was emulsified in complete Freund's adjuvant and injected subcutaneously into goats, at 0.5 mg of peptide per injection for the first two injections, followed at 2-week intervals by booster injections in the same adjuvant but with only 0.25 mg of peptide.

3. Isolation of Immunoglobulins (IgG) Specific of Total Apo-B and of Apo-B100

IgG were prepared with the modified protocol of Ritchie et al. (Ritchie 1972). Non-immunoglobulin proteins were eliminated from the immune serum and the IgG were dialyzed and concentrated.

The antibodies can be stored for example at 2 to 8° C. when used within the week, or frozen at −20° C. for example for prolonged use up to one month. The immunoglobulins contain sodium azide.

4. Antibody Specificity

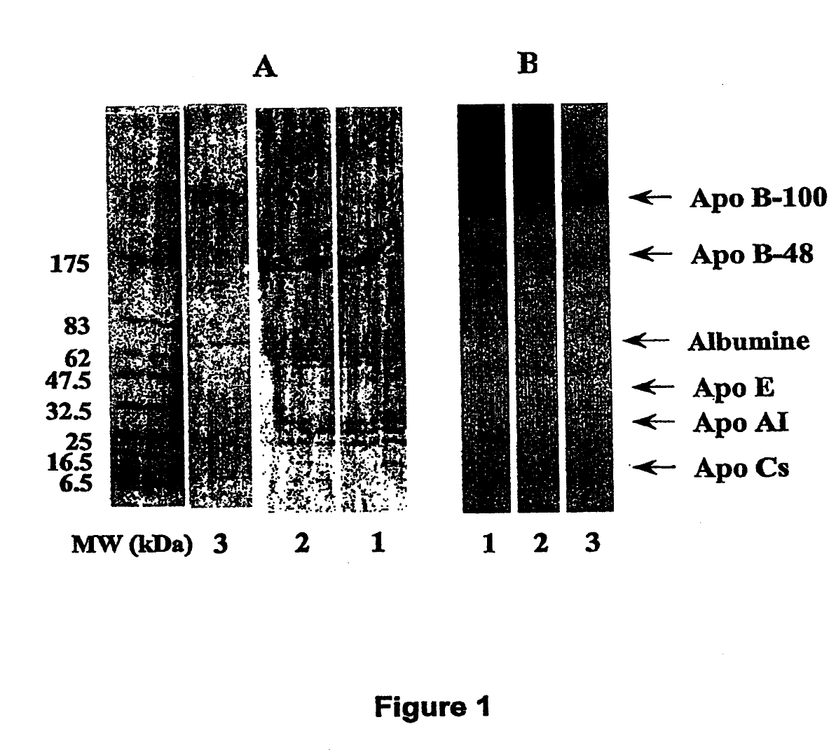

An analytical immunoblot of chylomicrons and VLDL was performed to demonstrate the specificity of the anti-Apo-B100 antibodies. As shown in FIG. 1, no cross reaction was observed with Apo-B48 or with other chylomicron proteins. As concerns VLDL, the anti-Apo-B100 antibody recognized only the protein located at 550 kDa which corresponds to the molecular mass of Apo-B100. These results demonsrate that the antibody directed against the peptide with SEQ ID NO: 1 specifically recognizes Apo-B100.

5. Immuno-Nephelometric Method of Analysis

5.1 Reagents and Materials used for the Nephelometric Analytical Method

TABLE 1

| | Name | | References |
|---|---|---|---|
| Anti-Apo-B | 1 × 6.5 ml | (300 tests) | Laboratory batch |
| immunoglobulin | | | |
| Anti-Apo-B100 | 1 × 5 ml | (100 tests) | Laboratory batch |
| immunoglobulin | | | |
| Standard | 3 × 0.5 ml | (900 tests) | Dade-Behring Ref. |
| | | | OUPG07 |
| Control | 3 × 0.5 ml | (500 tests) | Dade-Behring Ref. |
| | | | OUPH07 |
| Buffer 1 | 4 × 120 ml | (4 × 330 tests) | Beckman Coulter Ref. |
| | | | 447650 |
| IMMAGE UDR | 10 | (10 × 300 tests) | Beckman Coulter Ref. |
| cartridge | | | 447250 |
| Microtubes | 1000 | | Beckman Coulter Ref. |
| | | | 448162 |

Standard: The Apo-B standard was from a human serum calibrated with an electro-immunodiffusion (EIA) method and tested for HIV and hepatitis viruses. Said standard must be handled with the usual precautions so as to avoid any contamination. The Apo-B standard had a concentration of 100 mg/dl.

Preparation: To prepare a calibration curve, the standard was diluted in buffer 1 as follows.

TABLE 2

| Points | Concentration (mg/dl) |
|---|---|
| 1 | 0.156 |
| 2 | 0.312 |
| 3 | 0.6250 |
| 4 | 1.25 |
| 5 | 3.33 |
| 6 | 5 |
| 7 | 10 |
| 8 | 15 |
| 9 | 20 |

Storage and stability: The Apo-B standard can be stored and conserved at −20° C. EDTA and sodium azide are the stabilizers. The preparation can be frozen and dried.

Preparation of samples: Fresh samples are recommended for the analysis. Sera must be collected by established procedures used in clinical laboratories. If necessary, the serum can be stored at 2-8° C. for up to one week. Samples that are stored frozen can be used for longer periods: frozen samples are stable for up to one year.

Preparation of samples free of Apo-B100 particles: The following were added to a test tube in the stated order: 40 μl of anti-Apo-B-100 antibody, 40 μl of serum and 40 μl of buffer 1. The mixture was shaken and incubated for 10 minutes at room temperature, then centrifuged at 3500 rpm for 10 minutes after which the supernatant was removed for analysis. The final Apo-B48 concentration in the samples depleted of Apo-B100-containing lipoparticles was corrected for the supernatant dilution.

5.2 Protocol:
Program a reagent defined with the parameters listed in the IMMAGE immunochemical system instruction manual,
Transfer the reagent containing the antibody to compartment A of the cartridge,
Enter the value of the standard (the value of the current Apo-B standard is 1 mg/dl) in a parameter table, according to the dilutions shown in Table 2,
Use buffer 1 to dilute the sample.

5.3 Summary:

| | |
|---|---|
| Chemical name: Apo-B | Unit: mg/dl |
| Batch number: | Protocol: |
| See cartridge | Non-competitive nephelometry |
| Reaget Serial: | Expiration of reagent: |
| See cartridge | To be defined by user |
| Sample or dilution volume: 20 μl | Gain: 3 |
| Reagent buffer: 200 μl | Dilution: 1/1 |
| Volume of reagent compartment: 20 μl | Sample dilution: |
| | 1/15* |
| | 1/1** |
| Volume of compartment B: 0 μl | Reaction time: 2 minutes |

*to be configured after calibration.
**to be configured after Apo-B48 measurement.

5.4 Results

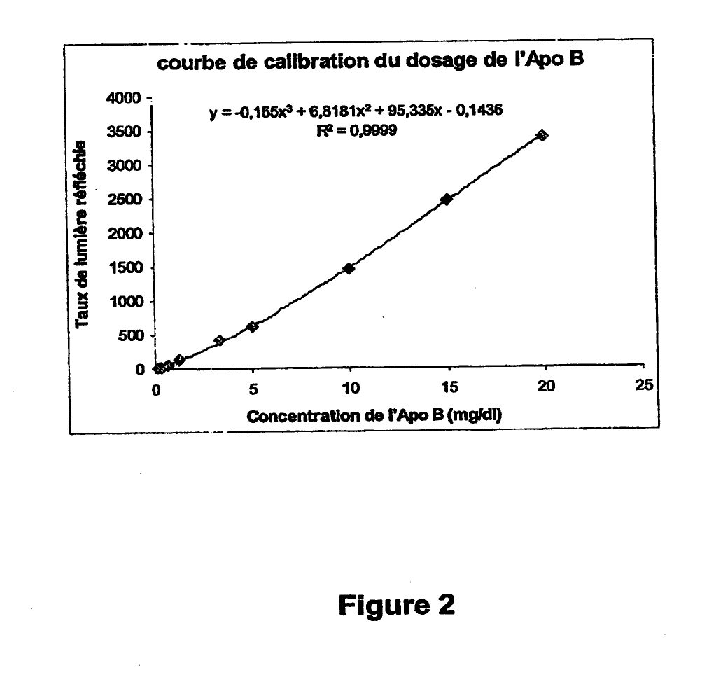

Calibration curve (FIG. 2): the calibration curve was plotted automatically by the analyzer.

Quality control: it is recommended to use a control serum (such as the serum from Dade-Behring GmbH, Marburg, Germany; for control of apolipoproteins) for each sample tested.

Values: the analyzer directly displays the final concentration. For treated samples containing anti-Apo-B100 antibody, it is necessary to correct for the three-fold dilution.

Working range:
For total Apo-B: 25 to 300 mg/dl
For Apo-B48 measurement: 1.5 to 25 mg/dl
For Apo-B100 measurement: difference between total Apo-B and Apo-B48.

Coefficient of variation: 4%

Usual values (Smith 1997):

Apo-B48:
4-6 mg/dl for a normal fasting subject
20 mg/dl postprandial value

6 Other Advantages of the Invention.

Other advantages and uses of the synthetic peptides specific of Apo-B100 of the invention include:
production of monoclonal antibodies,
use, as standard, for calibrating any Apo-B100 analytical method (ELISA, RIA, electroimmunodiffusion, etc.),
use in studies on different pathways of lipoprotein metabolism, to record or inhibit uptake of Apo-B-containing lipoproteins by LDL receptors.

Other advantages and uses of the inventive antibodies are as follows:
use in any immunological analytical method for differential quantification of Apo-B100 and Apo-B48,
use for differential detection of Apo-B100 and Apo-B48 (immunoblot, dot blot, immunohistochemistry and immunocytochemistry),
use in immunoaffinity and immunoprecipitation methods of protein purification.

### REFERENCES

Bassiri, R. M., Dvorak J. and Utiger R. D. (1979). Thyrotrpin-releasing hormone. Methods of hormone radioimmunoassay. In: Jaffe, B. M. and Behrman H. R. (Eds). New York, N.Y. Academic Press: p. 46.
Davidson, N. O., S. Anant, et al. (1995). “Apolipoprotein B messenger RNA editing: insights into the molecular regulation of post-transcriptional cytidine deamination.” Curr Opin Lipidol 6(2): 70-4.
Fievet, C., Koffigan M., Ouvry D., Marcovina S., Moschetto Y., Fruchart J. C. (1984). “Noncompetitive enzyme-linked immunoassay for apolipoprotein B in serum.” Clin Chem 30: 98-100.
Goldstein, J. L. and M. S. Brown (1977). “The low-density lipoprotein pathway and its relation to atherosclerosis.” Annu Rev Biochem 46: 897-930.
Harlow, E. a. L., D., Ed. (1988). Antibodies: a Laboratory Manual. New York, Cold Spring Harbor Laboratory Publications.
Havel, R. J., and Kane, J. P. (1995). Introduction: structure and metabolism of plasma lipoproteins. The Metabolic and Molecular Bases of Inherited Disease. A. L. B. C. R. Scriver, W. S. Sly, and D. Valle. New York, McGraw-Hill. 2: 1841-1851.
Hidaka, H., H. Kojima, et al. (1990). “Apolipoprotein B-48 analysis by high-performance liquid chromatography in VLDL: a sensitive and rapid method.” Clin Chim Acta 189(3): 287-96.
Hokanson, J. E. and M. A. Austin (1996). “Plasma triglyceride level is a risk factor for cardiovascular disease independent of high-density lipoprotein cholesterol level: a meta-analysis of population-based prospective studies.” J Cardiovasc Risk 3(2): 213-9.
Jones P. T., Dear P. H., Foote J., Neuberger M. S. and Winter G. (1986). “Replacing the complementarity-determining regions in a human antibody with those from a mouse.” Nature 321: 522-525.
Kane, J. P. (1983). “Apolipoprotein B: structural and metabolic heterogeneity.” Annu Rev Physiol 45: 637-50.
Karpe, F. and A. Hamsten (1994). “Determination of apolipoproteins B-48 and B-100 in triglyceride-rich lipoproteins by analytical SDS-PAGE.” J Lipid Res 35(7): 1311-7.
Kohler, G. and C. Milstein (1975). “Continuous cultures of fused cells secreting antibody of predefined specificity.” Nature 256(5517): 495-7.
Levinson, S. S. and S. G. Wagner (1993). “Immunonephelometric/turbidimetric apolipoprotein B assays for the clinical laboratory.” Clin Chim Acta 223(1-2): 31-42.
Li, B., Z. Wu, et al. (1997). “[Quantitation of human apolipoprotein B100 by immunoturbidimetric assay].” Hua Xi Yi Ke Da Xue Xue Bao 28(1): 109-12.
Merrifield, R. B. (1963). “Solid phase peptide synthesis. The synthesis of a tetrapeptide.” J. Am. Chem. Soc 85: 2149-2154.
Proctor, S. D. and J. C. Mamo (1997). “Separation and quantification of apolipoprotein B-48 and other apolipoproteins by dynamic sieving capillary electrophoresis.” J Lipid Res 38(2): 410-4.
Riechmann, L., Clark M., Waldmann H. and Winter G. (1988). “Monoclonal antibody therapeutic trials in seven patients with T-cell lymphoma.” Nature 332: 323-327.
Ritchie, R. F., and J. Stevens (1972). Qualifications for acceptable anti-serum performance in the automated immunoprecipitation system: A brief review of commercially available reagents. In: Advances in Automated Analysis. Technicon Symposia. Tarytown, N.Y., Mediad Inc: 9-14.
Simons, L. A., T. Dwyer, et al. (1987). “Chylomicrons and chylomicron remnants in coronary artery disease: a case-control study.” Atherosclerosis 65(1-2): 181-9.
Smith, D., Proctor S. D., Mamo J. C. (1997). “A highly sensitive assay for quantitation of apolipoprotein B48 using an antibody to human apolipoprotein B and enhanced chemiluminescence.” Ann Clin Biochem. 34:185-189.
Smith, D., S. D. Proctor, et al. (1997). “A highly sensitive assay for quantitation of apolipoprotein B48 using an antibody to human apolipoprotein B and enhanced chemiluminescence.” Ann Clin Biochem 34(Pt 2): 185-9.
Uchida, Y., Y. Kurano, et al. (1998). “Establishment of monoclonal antibody against human Apo B-48 and measurement of Apo B-48 in serum by ELISA method.” J Clin Lab Anal 12(5): 289-92.
Vaitukaitis, J., J. B. Robbins, et al. (1971). “A method for producing specific antisera with small doses of immunogen.” J Clin Endocrinol Metab 33(6): 988-91.
Wagner, H. A., R. Nustede, et al. (1987). “Isolation of apolipoprotein-B-48 from chylomicrons by high-performance liquid chromatography.” J Chromatogr 397: 405-8.
Ward, E. S., D. Gussow, et al. (1989). “Binding activities of a repertoire of single immunoglobulin variable domains secreted from Escherichia coli.” Nature 341(6242): 544-6.
Young, S. G. (1990). “Recent progress in understanding apolipoprotein B.” Circulation 82(5): 1574-94.

1-33. (canceled)

34. A substantially pure synthetic peptide, comprising the amino acid sequence SEQ ID NO: 1 or SEQ ID NO: 2 or an immunogenic fragment or a derivative of said sequences.

35. A substantially pure synthetic peptide according to claim 34, comprising the amino acid sequence SEQ ID NO: 1 or SEQ ID NO: 2.

36. The peptide according to claim 34, wherein the peptide is soluble or complexed with a carrier molecule such as KLH, serum albumin or a bead.

37. The peptide according to claim 34, wherein it allows the production of ApoB100-immunoprecipitant antibodies.

38. An antibody specific of a synthetic peptide, comprising the amino acid sequence SEQ ID NO: 1 or SEQ ID NO: 2 or an immunogenic fragment or a derivative of said sequences, or fragment or derivative of said antibody having substantially the same antigenic specificity.

39. The antibody according to claim 38, wherein it is produced by immunizing a non-human animal with the peptide, and recovering the antibodies or the antibody-producing cells.

40. The antibody according to claim 38, wherein it is a polyclonal antibody.

41. The antibody according to claim 38, wherein it corresponds to a mixture of monoclonal antibodies.

42. The antibody according to claim 38, wherein it is capable of precipitating Apo-B100.

43. A method for producing a specific anti-Apo-B100 antibody, comprising administering a peptide according to claim 34 or a mixture thereof to a non-human animal and recovering the antibodies or antibody-producing cells.

44. A method for detecting or quantifying Apo-B100 in a biological sample, comprising contacting the sample with an antibody specific to a peptide according to claim 34 or with an antibody produced by a method according to claim 43, and detecting the presence of antigen-antibody immune complexes.

45. A method for detecting or differentially quantifying Apo-B100 and Apo-B48 in a sample, comprising the following steps:
(a) determining the amount of total Apo-B in a sample with a total anti-Apo-B antibody,
(b) eliminating Apo-B100 or lipoparticles containing Apo-B100 from said sample, with the help of a specific anti-Apo-B100 antibody specific of a peptide according to claim 34 or produced by a method according to claim 43, and
(c) determining the amount of Apo-B48 in the sample obtained after step (b) or in lipoparticles not containing Apo-B100 in said sample, with a total anti-Apo-B antibody.

46. The method according to claim 45, wherein, during steps (a) and/or (c), a polyclonal antibody directed against total anti-Apo-B is used.

47. The method according to claim 45, wherein the specific anti-Apo-B100 antibody used in step (b) is a polyclonal antibody or a mixture of monoclonal antibodies or a modified antibody allowing precipitation of immune complexes.

48. The method according to claim 45, wherein the specific anti-Apo-B100 antibody used in step (b) is an immunoprecipitant polyclonal antibody.

49. The method according to claim 45, wherein the presence of an antigen-antibody immune complex is determined by ELISA, RIA, EIA, turbidimetry or any other immunological assay.

50. The method according to claim 45, wherein the presence of an antigen-antibody immune complex is determined by a nephelometric analytical method.

51. A method for detecting or differentially quantifying Apo-B100 and Apo-B48 in a biological sample comprising contacting said sample with an antibody specific of a peptide according to claim 34 or with an antibody produced by a method according to claim 43, and indirectly detecting the formation of Apo-B100-antibody immune complexes by a nephelometric analytical method.

52. The method according to claim 45, wherein the biological sample is a sample of blood or plasma or serum or interstitial fluid or cerebrospinal fluid or a cell culture supernatant.

53. A method for detecting the presence, a predisposition or a risk of developing a disorder of lipid metabolism in a subject, comprising differentially quantifying in vitro the amounts of Apo-B48 and Apo-B100 in a sample from a subject, with an antibody specific of a peptide according to claim 34, or with an antibody produced by a method according to claim 43.

54. The method according to claim 53, for detecting the presence, a predisposition or a risk of developing atherosclerosis or a cardiovascular disease such as a coronary disease for example.

55. A method for monitoring a treatment for correcting disorders of lipid metabolism in a subject, comprising quantifying Apo-B100 and/or Apo-B48 in vitro, in a sample from said subject, with the help of an antibody specific of a peptide according to claim 34 or an antibody produced by a method according to claim 43.

56. The method according to claim 55, for monitoring a treatment of atherosclerosis or of a cardiovascular disease such as a coronary disease for example.

57. A method for detecting or monitoring the cellular uptake of LDL and triglyceride-rich lipoproteins in a subject, comprising detecting in vitro the amounts of Apo-B48 and/or Apo-B100 present in lipoparticles, with the help of an antibody specific of a peptide according to claim 34 or an antibody produced by a method according to claim 43.

58. A method for evaluating the physiological state of a subject, comprising detecting the levels of Apo-B48 and Apo-B100 in vitro in a sample from said subject, with the help of an antibody specific of a peptide according to claim 34 or an antibody produced by a method according to claim 43.

59. A method for screening compounds or diets capable of modulating the serum or plasma concentration of Apo-B48 and/or Apo-B100, by implementing an antibody specific of a peptide according to claim 34 or produced by a method according to claim 43.

60. The antibody specific of a peptide according to claim 34 or produced by a method according to claim 43, wherein the antibody is coupled with heterologous fragments such as toxins, labels, medicaments or any other therapeutic agent.

61. A method for selectively regulating Apo-B100 activity in vivo, by administering into a subject in need of such treatment an antibody specific of a peptide according to claim 34 or produced by a method according to claim 43.

62. A pharmaceutical composition comprising an antibody specific of a peptide, comprising the amino acid sequence SEQ ID NO: 1 or SEQ ID NO: 2 or an immunogenic fragment or a derivative of said sequences, or an antibody produced by a method according to claim 43, and a pharmaceutically acceptable vehicle or excipient.

63. A method for screening compounds modulating Apo-B activity by implementing a substantially pure synthetic peptide, comprising the amino acid sequence SEQ ID NO: 1 or SEQ ID NO: 2 or an immunogenic fragment or a derivative of said sequences.

64. A method for specifically modulating Apo-B100 activity, by implementing a substantially pure synthetic peptide comprising the amino acid sequence SEQ ID NO: 1 or SEQ ID NO: 2 or an immunogenic fragment or a derivative of said sequences.

65. A kit comprising a substantially pure synthetic peptide comprising the amino acid sequence SEQ ID NO: 1 or SEQ ID NO: 2 or an immunogenic fragment or a derivative of said sequences, or an antibody specific of said peptide or an antibody produced by a method according to claim 43.
